(19) United States
(12) Patent Application Publication
(10) Pub. No.: US 2011/0268769 A1
(43) Pub. Date: Nov. 3, 2011

(54) DRUG DELIVERY VEHICLE FOR CANCER THERAPY, PROCESS FOR PRODUCING THE SAME, AND PHARMACEUTICAL PREPARATION USING THE SAME

(76) Inventors: Yasufumi Kaneda, Osaka (JP); Chun-Man Lee, Osaka (JP); Yasuhiko Tabata, Kyoto (JP); Tomoyuki Asano, Osaka (JP)

(21) Appl. No.: 13/103,275

(22) Filed: May 9, 2011

Related U.S. Application Data
(63) Continuation of application No. 12/054,200, filed on Mar. 24, 2008, now abandoned.

(30) Foreign Application Priority Data
Jun. 14, 2007 (JP) 2007-157701

(51) Int. Cl. A61K 9/00; A61K 31/69; A61K 38/00; A61P 35/00; A61K 47/36; A61K 31/7088

(52) U.S. Cl. 424/400; 514/777; 514/44 R; 514/19.3; 514/64

(57) ABSTRACT

The invention provides a vehicle that can deliver drugs specifically to the body and a pharmaceutical preparation using the same. Disclosed is a drug delivery vehicle for cancer therapy, comprising a cationized viral envelope vector, as well as a pharmaceutical preparation comprising a drug enclosed in the vehicle. The viral envelope vector is for example HVJ-E derived from a Sendai virus, and cationization can be conducted by binding hyaluronic acid-introduced cationized gelatin or ethylene glycol-introduced cationized gelatin with the viral envelope vector. The drug to be enclosed is a nucleic acid, a vector containing a nucleic acid sequence, a protein based drug or pharmaceutical with a low-molecular compound.

### BACKGROUND OF THE INVENTION

1. Field of the Invention

The present invention relates to a drug delivery vehicle for cancer therapy, a process for producing the same, and a pharmaceutical preparation using the same. The present invention relates in particular to a drug delivery vehicle for cancer therapy using a viral envelope vector, a process for producing the same, and a pharmaceutical preparation using the same.

2. Description of the Related Art

For introduction of a gene mainly into a specific site in the living body, an introduction method using a virus and (synthetic) non-virus method has been developed. For example, introduction of a gene with a viral vector derived from an adenovirus and introduction of a gene with a liposome are known, but the viral vector has problems such as toxicity and concern about pathogenicity, while the non-viral vector has a problem of low introduction efficiency.

To solve such problems, an HVJ envelope vector (HVJ-E) utilizing an envelope obtained by inactivating a Sendai virus has been developed, focusing attention on an excellent membrane fusion ability of Sendai virus, thereby solving problems such as pathogenicity and toxicity (WO 01/57204). Since membrane fusion of the HVJ-E enables the direct transfer (or introduction) of an intended substance into cytoplasm, the substance is hardly decomposed and thus retain its functions. Accordingly, this envelope vector can be effective, for example, in treatment of allergic rhinitis by protein introduction and in treatment of cancer with siRNA.

The disadvantage of the HVJ envelope vector is that this vector fuses with almost all types of cells except for peripheral lymphocytes. For enhancing the delivery specificity of HVJ-E, development of a targeting vector by further modification of HVJ-E and a gene or drug delivery system using the same have also been made (Mima et al., Mol. Cancer Ther. 5(4); 1021-8, 2006, April).

### SUMMARY OF THE INVENTION

With respect to the drug delivery system using HVJ-E, however, its in vivo behavior, its method of reliably delivering a drug to a target site and its mechanisms are not fully elucidated. Delivery of a targeting vector which is derived from further modification of previously developed HVJ-E or a gene or drug delivery system using such vector is ensured sometimes by limiting the route of administration. Accordingly, there is a strong demand for a system that regulates in vivo behavior of an intended drug and delivers the drug efficiently, specifically and easily to a target.

Accordingly, one of the objectives of the present invention is to provide a delivery system for delivering an intended drug specifically to a desired cell or tissue.

The present inventors made extensive study, and as a result, they found that the objective can be achieved by providing the following drug delivery vehicle for cancer therapy and a process for producing the same, thereby arriving at completion of the present invention.

That is the present invention provides a drug delivery vehicle for cancer therapy, comprising cationized gelatin having hyaluronic acid and/or polyethylene glycol bound thereto and a viral envelope vector.

The viral envelope vector can be HVJ-E derived from a Sendai virus.

The present invention also provides a pharmaceutical preparation comprising a drug enclosed in the drug delivery vehicle for cancer therapy.

The drug can be selected from the group consisting of a small molecular compound, a nucleic acid, a nucleic acid-containing plasmid vector, and a protein based drug.

The drug can be an antitumor agent.

The antitumor agent can be at least one member selected from the group consisting of cyclophosphamide, mechlorethamine, carbazylquinone, melphalan, teotepa, busulfan, nimustine, carmustine, procarbazine, dacarbazine, methotrexate, 6-mercaptopurine, 6-thioguanine, azathioprine, 5-fluorouracil, phthraful, floxuridine, cytarabine, ancitabine, tegafur, doxifluridine, actinomycin D, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, etoposide, carboplatin, estramustine phosphate, mitotane, porphyrin, and taxol.

The drug for cancer therapy can be a boron-containing compound.

The boron-containing compound can be mercaptoundecahydrododecaborate (BSH) or p-boronophenylalanine (BPA).

The pharmaceutical preparation containing the boron-containing compound can be used in boron neutron capture therapy (BNCT).

The pharmaceutical preparation can be used in therapy of one member selected from malignant pleural mesothelioma and hepatoma.

The present invention also provides a process for producing the drug delivery vehicle for cancer therapy described above, comprising:

(a) a step of inactivating a virus, and
(b) a step of cationizing a viral envelope vector obtained from the inactivated virus, with hyaluronic acid and/or polyethylene glycol, a cationizing agent, and gelatin.

Further, the present invention provides a process for producing the drug delivery vehicle for cancer therapy described above, comprising:

(a) a step of inactivating a virus, and
(b) a step of binding cationized gelatin having hyaluronic acid and/or polyethylene glycol bound thereto, with a viral envelope vector obtained from the inactivated virus.

The viral envelope vector can be HVJ-E derived from a Sendai virus.

According to the present invention, there can be provided a delivery system capable of delivering a drug for cancer therapy safely and highly specifically to a desired cell or tissue by multiple or any given administration routes. The drug delivery vehicle for cancer therapy according to the present invention has a cancer inhibitory action and/or an immunoenhancing action by itself and thus has a very potent effect together with the action of a drug enclosed therein.

### BRIEF DESCRIPTION OF THE DRAWINGS

FIG. 1 is a graph showing examination results of the efficiency of introduction of a luciferase gene with polymer-bound HVJ-E.

FIG. 2 shows the proliferation rate of a tumor irradiated in vitro with neutrons by using polymer-bound HVJ-E having BSH enclosed therein.

FIG. 3 is a graph showing an inhibitory effect on hepatic metastasis in neutron capture therapy by using polymer-bound HVJ-E having BSH enclosed therein.

### DETAILED DESCRIPTION OF THE INVENTION

Hereinafter, embodiments of the present invention are described in detail.

The terms used in this specification are described. In this specification, the term “virus” is an infectious microstructure having DNA or RNA as genome and proliferating in only infected cells. The virus includes viruses belonging to a family selected from the group consisting of Retroviridae, Togaviridae, Coronaviridae, Flaviviridae, Paramyxoviridae, Orthomyxoviridae, Bunyaviridae, Rhabdoviridae, Poxyiridae, Herpesviridae, Baculoviridae and Hepadnaviridae. A virus used preferably in the present invention is a Sendai virus (HVJ (hemagglutinating virus of Japan)) belonging to the genus paramyxovirus in Paramyxoviridae. The genome of Sendai virus is a negative-strand RNA having a base length of about 15500. The virus particle possesses a polymorphic envelope with a diameter of 150 to 300 nm.

The “(viral) envelope” used herein refers to a membrane structure based on a lipid bilayer surrounding a nucleocapsid present in a specific virus such as Sendai virus. The envelope is observed usually in a virus matured by sprouting from a cell. The envelope is composed generally of a small projected structure consisting of a spike protein encoded by a viral gene and a lipid derived from a host. The “viral envelope vector” refers to a vector having an exogenous gene enclosed in a viral envelope, but a viral envelope in which no exogenous gene is enclosed is also referred to a viral envelope vector in this specification in the sense that it can carry a drug.

Viral “inactivation” used herein refers to inactivation of a genome of a virus (for example, Sendai virus). The inactivated virus is impaired in its replication function, but keeps its viral fusion ability.

“The drug delivery vehicle for cancer therapy” used herein refers to a vehicle that can enclose a drug for cancer therapy inside of a viral envelope, preferably a vehicle of which outer membrane is cationized. The drug delivery vehicle for cancer therapy according to the present invention has a cancer inhibitory action and/or an immunoenhancing action by itself, and thus a pharmaceutical preparation using this vehicle has a very potent cancer inhibitory effect together with the action of a drug enclosed therein.

“Cationization” used herein refers to that which gives a positive charge to a certain object, and refers herein to preparation of a viral envelope by positively charging the outer side of a membrane, or to the state of a material giving a positive charge so as to achieve such preparation. Specifically, cationization refers to contacting a cationic polymer such as hyaluronic acid-bound (or -introduced) cationized gelatin (CG-HA) or polyethylene glycol-bound (or -introduced) cationized gelatin (CG-PEG), with the outer side of a viral envelope membrane or to the state of such cationized polymer. This contacting results, for example, in achievement including, but not limited to, formation of an electrostatic bond, In this specification, the term “bond” or “bound to” refers also to the state of a cationic polymer retained by some sort of action on the outer surface of the membrane, even without a formation of electrostatic bond.

As used herein, “cationized gelatin” (CG) can be obtained for example by treating gelatin of a relatively low molecular weight (for example, about 3,000 to about 5,000) with a cationizing agent having a reactive group which binds directly to a carboxyl group present in the gelatin. As used herein, the cationizing agent is a compound having both a moiety capable of generating a cation and a reactive group capable of binding with a carboxyl group present in gelatin. Such compound includes, but is not limited to, ethylene diamine. For example, ethylene diamine can be reacted with a carboxyl group in gelatin to form an amido linkage to allow the gelatin to have an amino group, thereby giving cationized gelatin.

In this specification, “hyaluronic acid-bound (or -introduced) cationized gelatin” (CG-HA) is obtained by further reacting hyaluronic acid with the cationized gelatin. Though not intended to be limitative, CG-HA can also be obtained by generating a reaction between a sugar reducing terminal of hyaluronic acid and an amino group.

In this specification, “polyethylene glycol-bound (or -introduced) cationized gelatin” (CG-PEG) is obtained by reacting polyethylene glycol with the cationized gelatin. By adding CG-HA and CG-PEG to a viral envelope, there is brought about high affinity particularly for a cancer cell or a stealth effect.

The present invention also provides a pharmaceutical preparation comprising an intended drug for cancer therapy enclosed in a viral envelope vector cationized with hyaluronic acid-bound (or -introduced) cationized gelatin or polyethylene glycol-bound (or -introduced) cationized gelatin.

The intended drug for cancer therapy is not limited, and may be any drug known in the art, Such drug includes a DNA or RNA encoding a protein having a specific function that can be used in gene therapy, a DNA or RNA not encoding a protein having a specific function, a vector containing the same; various proteins (for example, protein based drugs including an antigen, an antibody and an enzyme); boron-containing compounds that can be used in neutron capture therapy; and antitumor agents.

The boron-containing compounds include, but are not limited to, those preferably used at present in boron neutron capture therapy and known boron-containing compounds such as a boron ligand-bound dendrimer having at least one boron ligand bound to a dendrimer (JP-A 2006-96870). Boron neutron capture therapy (BNCT) is cancer therapy attracting attention at present. In boron neutron capture therapy, a boron compound containing a ¹⁰boron isotope (¹⁰B) is incorporated into a cancer Cell and is then irradiated with a low-energy neutron ray (for example, thermal neutron) to break the cancer cell locally by a nuclear reaction occurring in the cell. In this therapeutic method, the selective accumulation of a ¹⁰B-containing boron compound in cells of cancer tissue is important in enhancing the therapeutic effect, and thus boron compounds to be incorporated selectively into cancer cells are developed.

Boron-containing compounds having a boron atom or a boron atomic group introduced into their basic skeleton have been synthesized as drugs used in BNCT. Clinically used drugs include p-boronophenylalanine (BPA) and mercaptoundecahydrododecaborate (BSH). Among these drugs, BSH is used in the form of a sodium salt mainly for treatment of a brain tumor and confirmed to be useful (for example, I. M. Wyzlic et al., Tetrahedron Lett., 1992, 33, 7489-7490, W. Tjark, J. Organomet. Chem., 2000, 614-615, 37-47; K. Imamura et al., Bull. Chem. Soc. Jpn., 1997, 70, 3103-3110; A. S. Al-Madhorn et al., J. Med. Chem., 2002, 45, 4018-4028; F. Compostella et al., Res. Develop. Neutron Capture Ther., 2002, 81-84; S. B. Kahl et al., Progress in Neutron Capture Therapy for Cancer, Plenum Press, New York 1992, 223; J. Cai et al., J. Med. Chem., 1997, 40, 3887-3896; H. Lim et al., Res. Develop. Neutron Capture Ther., 2002, 37-42).

The antitumor drug includes, but is not limited to, at least one member selected from the group consisting of cyclophosphamide, mechlorethamine, carbazylquinone, melphalan, teotepa, busulfan, nimustine, carmustine, procarbazine, dacarbazine, methotrexate, 6-mercaptopurine, 6-thioguanine, azathioprine, 5-fluorouracil, phthraful, floxuridine, cytarabine, ancitabine, tegafur, doxifluridine, actinomycin D, bleomycin, mitomycin, chromomycin A3, cinelbin A, aclacinomycin A, adriamycin, peplomycin, mitoxantrone, epirubicin, pirarubicin, vinblastine, vincristine, vindesine, etoposide, cisplatin, carboplatin, estramustine phosphate, mitotane, porphyrin, and taxol or a combination thereof.

In the present invention, the drug for cancer therapy may be suitably combined with another drug if necessary and contained in one drug delivery vehicle for cancer therapy. The other drug includes, but is not limited to, central nervous system drugs (for example, a general anesthetic, a hypnotic/analgesic agent, an antianxiety drug, an antiepileptic drug, an antipyretic analgesic antiflash agent, an analeptic remedy, a psychostimulant, an antiparkinson agent, a psychoneurotic agent, a multi-symptom cold remedy, other agents affecting the central nervous system, etc.); peripheral nerve drugs (for example, a local anesthetic, a skeletal muscle relaxant, an autonomic agent, a spasmolytic agent etc.); sense organ drugs (for example, ophthalmologic drug, an otologicali agent, an antidinic agent etc.); circulatory drugs (for example, a cardiotonic agent, an antiarrhythmic agent, a diuretic agent, a hypotensive agent, a vasoconstrictor, a vasodilator, a lipid-lowering drug, other circulatory drugs); respiratory drugs (for example, a respiratory stimulant, an antitussive agent, an expectorant, an antitussive expectorant, a bronchodilator etc.); digestive drugs (for example, an antiemetic drug, an antiflatulent, a stomachic digestive drug, an antacid, a cholagogue, other digestive drugs etc.); hormonal agents (for example, a hypophysis hormone, a salivary gland hormone, a thyroid hormone, a parathyroid hormone, an anabolic steroid hormone, an adrenal hormone, an androgenic hormone, a mixed hormone, other hormones etc.) urogenital and anal drugs (for example, an urinary agent, a genital agent, an oxytocic agent, a hemorrhoidal agent, other urogenital and anal drugs, etc.); dermatologic preparations (for example, an antimicrobial for external use, a wound protective agent, a purulent disease agent, an analgesic, an antipruritic, an astringent, an antiphlogistic, a parasitic skin disease, a skin emollient, a preparation for the hair, other dermatologic preparations etc.); agents for dental and oral use; other drugs for individual organ system; vitamin preparations (for example, vitamin A, vitamin D, vitamin B, vitamin C, vitamin E, vitamin K, a mixed vitamin, other vitamins etc.); analeptics (for example, a calcium preparation, a mineral preparation, a sugar preparation, a protein amino acid preparation, an organ preparation, agents for infants, other analeptics etc.); blood and body fluid agents (for example, a blood replacement fluid, a hemostatic drug, a blood coagulation inhibitor, other blood and body fluid agents, etc.); other metabolized pharmaceuticals (for example, an organ disease drug, an antidote, an agent for habitual addiction, a gout remedy, an enzyme preparation, a diabetic drug, other unclassified metabolized drugs etc.); cellular stimulants (for example, a chlorophyll preparation, a pigment preparation, other cell stimulants etc.); allergy drugs (for example, an antihistamine, an agent for stimulation therapy, a nonspecific immunogen, other allergy drugs, pharmaceuticals based on herbal medicine and Chinese medicine formulation, a herbal medicine, a Chinese medicine, other pharmaceuticals based on herbal medicine and Chinese medicine formulation, etc.); antibiotics preparations (for example, a drug acting on Gram-positive bacteria or Gram-negative bacteria, a drug acting on a Gram-positive bacterium mycoplasma, a drug acting on Gram-positive or Gram-negative rickettsia, a drug acting on acid-fast bacilli, a drug acting on molds, other antibiotics preparations, etc.); chemotherapeutic drugs (for example, a sulfa drug, an antituberculous drug, a synthetic antibacterial drug, an antiviral drug, other chemotherapeutic drugs, etc.); biological preparations (for example, a vaccine, toxoids, blood preparations, drugs for biological test, other biological preparations, an antiprotozoal agent, a vermifuge, etc.); prescription drugs (for example, an excipient, an ointment base, a solubilizer, a coloring agent, other prescription drugs, etc.); and narcotic drugs (for example, an opium alkaloid narcotic drug, a coca alkaloid preparation, a synthetic narcotic drug, etc.).

The pharmaceutical preparation of the present invention can be used not only in humans but also in other hosts as the target.

The process for producing the drug delivery vehicle for cancer therapy according to the present invention comprises (a) a step of inactivating a virus, and (b) a step of cationizing a viral envelope vector obtained from the inactivated virus, with hyaluronic acid and/or polyethylene glycol, a cationizing agent, and gelatin. The step of cationizing the viral envelope vector involves binding, for example, cationized gelatin having hyaluronic acid and/or polyethylene glycol bound thereto, with a viral envelope vector. The cationized gelatin having hyaluronic acid and/or polyethylene glycol bound thereto is obtained most generally by binding cationized gelatin obtained by treating gelatin with a cationizing agent having a reactive group binding directly with a carboxyl group of the gelatin, with hyaluronic acid or polyethylene glycol that is a mixture having various molecular weights.

The virus is used after suitable proliferation prior to preparation of the drug delivery vehicle for cancer therapy according to the present invention. For example, HVJ can be generally used after proliferation by inoculation of the seed virus into a hen fertilized egg, but HVJ proliferated from a strain persistently infecting (with a hydrolase such as trypsin added to a culture of) cultured cells or tissues such as simian or human cultured cells or tissues, or HVJ proliferated after infecting cultured cells persistently with its cloned viral genome, and all mutants thereof, can be used in the present invention. Viruses (for example, HVJ) available by other methods can also be used. Recombinant HVJ (Hasan M. K., et al., Journal of General Virology, 78, 2813-2830, 1997 or Yonemitsu Y., et al., Nature Biotechnology 18, 970-973, 2000) can also be used. Any HVJ strains may be used among which Z strain (for example, the virus available under Accession No. ATCC VA 2388 or from Charles River SPAFAS) or Cantell strain (for example, the virus described by M. D. Johnston in J. Gen. Virol., 56, 175-184, 1981 or available from Charles River SPAFAS) is more desired.

The method of inactivating the virus (for example HVJ) is not particularly limited. Such method includes known methods such as thermal treatment (for example, at 60° C. for 1 hour), ultraviolet (UV) ray irradiation, chemical treatment with chemicals such as phenol and formalin, freeze-thawing, and treatment with an alkylating agent.

Inactivation of the virus, for example HVJ, is evaluated by whether the infection of cultured cells with HVJ occurs or not. For example, viral inactivation can be evaluated by inactivating the virus and then infecting simian renal cell strain LLC-MK2 with the treated virus. Because one-step growth of HVJ occurs at 12 to 18 hours after infection, the cells after infection are incubated for 18 to 24 hours and then fixed with acetone/methanol, and whether protein F of HVJ expressed in the HVJ-infected cells occurs or not can be examined by immunostaining with an antibody to protein F. That is, HVJ is solubilized and centrifuged to separate a membrane component, and the resulting membrane component is subjected to ion-exchange chromatography to give protein F (according to Yoshima, H., et al., J. Biol. Chem. 1981, and Suzuki, K., et al., Gene Therapy and Regulation, 2000). Then, this protein F, together with Freund's adjuvant, is used to immunize a rabbit to give an antiserum to protein F (rabbit anti-protein F polyclonal antibody; primary antibody). The fixed cells are treated with this primary antibody and then treated with a secondary antibody. After treatment with this secondary antibody, the cells can be observed under a fluorescence microscope to evaluate the inactivation of HVJ. The influence of the inactivation treatment on the membrane function of the virus (for example HVJ) envelope can be examined by measuring, as an indicator, the HA activity of the inactivated virus (for example HVJ) envelope. The HA activity can be measured by a usual method. A suspension of the inactivated HVJ envelope is added to a well plate and then diluted serially to prepare serially diluted samples. Whether there is an agglutination reaction in these samples is examined. The HA activity is determined from the amount of the serially diluted sample in which an agglutination reaction is lost and a reciprocal of the dilution rate in the corresponding well.

The inactivated virus (for example HVJ) is purified by a method such as column chromatography, ultrafiltration, or a combination thereof. In column chromatography, both a weak anion exchanger (having exchange groups such as tertiary amine DEAE bound thereto) and a strong anion exchanger (having exchange groups such as quaternary amine QAE bound thereto) can be used. Column chromatography using a gel filtration carrier can also be used.

The inactivated virus (for example HVJ) envelope is cationized with hyaluronic acid-bound (or -introduced) cationized gelatin or polyethylene glycol-bound (or -introduced) cationized gelatin, whereby the drug delivery vehicle for cancer therapy according to the present invention can be obtained. This drug delivery vehicle for cancer therapy is useful in enclosing various drugs therein. Typically, the drug delivery vehicle for cancer therapy according to the present invention is mixed with a drug, whereby a pharmaceutical Preparation comprising the drug enclosed in the drug delivery vehicle for cancer therapy according to the present invention can be obtained.

Alternatively, the obtained inactivated virus (for example HVJ) is mixed as a “viral envelope vector” with a drug to prepare a drug-enclosed viral envelope vector complex which is then cationized by binding it with hyaluronic acid-bound (or -introduced) cationized gelatin or polyethylene glycol-bound (or -introduced) cationized gelatin, whereby a pharmaceutical preparation comprising the drug enclosed in the drug delivery vehicle for cancer therapy according to the present invention can be obtained.

Cationization of the viral envelope vector is not limited, but can be conducted by bringing the viral envelope vector into contact with hyaluronic acid-bound (or -introduced) cationized gelatin or polyethylene glycol-bound (or -introduced) cationized gelatin, thereby preferably forming an electrostatic bond.

The cationized gelatin can be obtained for example by mixing gelatin having a relatively low molecular weight with a cationizing agent such as ethylene diamine in a buffer under the conditions where a carboxyl group of the gelatin reacts with an amino group of the ethylene diamine, and then reacting them overnight at a temperature of about 25 to 40° C. in the presence of EDC (ethyl-3-(3-dimethylaminopropyl) carbodiimide hydrochloride). The resulting polymer can also be dialyzed and then dried.

Though not intended to be limitative, the hyaluronic acid-bound (or -introduced) cationized gelatin is prepared as follows: The cationized gelatin obtained for example as described above and hyaluronic acids having various molecular weights are added to a carbonate buffer. Then, the mixture is stored in the presence of a catalyst, thereby reacting a sugar reducing terminal with an amino group of the cationized gelatin. The resulting product is dialyzed and then dried to give the hyaluronic acid-bound (or -introduced) cationized gelatin. The hyaluronic acids having various molecular weights as used herein are obtained by thermally decomposing hyaluronic acid having a molecular weight of about 1,800,000, in, for example, an autoclave, and then dialyzing and purifying the product. For purification of the hyaluronic acid-bound cationic gelatin, hyaluronic acids having molecular weight of about 5,000 to 1,000,000 can be used, and hyaluronic acids having the respective molecular weights can be separated by the molecular weights and the degree of cationization as parameters, and used. The degree of cationization is the degree of introduction of amino groups into carboxyl groups of gelatin and is preferably 5 to 50%.

The hyaluronic acid:cationized gelatin ratio, in terms of molar ratio, is preferably from 10:1 to 1:10, more preferably from 2:1 to 1:2.

The polyethylene glycol-bound (or -introduced) cationized gelatin is another example that can be preferably used in the present invention. Though not intended to be limitative, the polyethylene glycol-bound (or -introduced) cationized gelatin is prepared as follows: Cationized gelatin obtained for example as described above and polyethylene glycols having various molecular weights are added to a carbonate buffer. Then, the mixture is stored in the presence of a catalyst, thereby reacting an aldehyde group at the terminal of the polyethylene glycol with an amino group of the cationized gelatin. The product thus obtained is dialyzed and then dried, to prepare the polyethylene glycol-bound (or -introduced) cationized gelatin. The molecular weights of the respective polyethylene glycols range from about 10 kDa (kilodalton) to 100 kDa. In another aspect, the molecular weights of the respective PEG molecules range from about 10 kDa to 40 kDa. In still another aspect, the molecular weights of the respective PEG molecules are about 12 kDa. In a further aspect, the molecular weights of the respective PEG molecules are about 20 kDa. Suitable PEG molecules can be obtained from Shearwater Polymers, Inc. and Enzon, Inc. and can be selected from SS-PEG, NPC-PEG, aldehyde-PEG, mPEG-SPA, mPEG-SCM, mPEG-BTC, SC-PEG, tolesylated mPEG (U.S. Pat. No. 5,880,255) and oxycarbonyl-oxy-N-dicarboximide-PEG (U.S. Pat. No. 5,122,614), but polyethylene glycols having molecular weights suitable for purification of the polyethylene glycol acid-bound cationized gelatin can be those having molecular weights of about 1,000 to 100,000, and polyethylene glycols having the respective molecular weights are separated by the molecular weights and the degree of introduction of polyethylene glycol (1 to 20%) as parameters, and used. The degree of introduction is an indicator showing the degree of introduction of polyethylene glycols into amino groups of cationized gelatin. The polyethylene glycol:cationized gelatin ratio, in terms of molar ratio, is preferably from 10:1 to 1:10, more preferably from 2:1 to 1:2.

The hyaluronic acid-bound (or -introduced) cationized gelatin or polyethylene glycol-bound (or -introduced) cationized gelatin obtained as described above (hereinafter referred to collectively as “various polymers”) is bound to the viral envelope vector. The viral envelope vector may be in a state before or after enclosing a drug therein as described above. The compounding ratio of the various polymers: HVJ-E is basically from 5 μg:1 HAU to 1 μg:5 HAU, more preferably 250 μg:500 HAU, and they are compounded in the following manner. That is, the various polymers are dissolved at a concentration of about 10 mg/ml to 50 mg/ml in PBS or the like as the solvent, and the HVJ-E stock is added thereto followed by pipetting. Further, a buffer such as PBS is added thereto, and the mixture is left on ice for a predetermined time to prepare the drug delivery vehicle.

Incorporation of a drug into the viral envelope vector or the drug delivery vehicle for cancer therapy is achieved specifically by dissolving the drug in a solvent to form a solution and mixing this solution with a solution of the viral envelope vector. For incorporation of the drug, a surfactant can also be preferably used. When the drug is for example cisplatin, the drug solution is mixed, in a ratio of from about 2 (HAU):1 (μg) to 1 (HAU):2 (μg) with the viral envelope vector to which the various polymers were bound or not bound. Then, a surfactant-containing buffer is added to the mixture which is then centrifuged, followed by removing a supernatant, to give a drug-enclosed preparation.

Incorporation of a boron-containing compound into the viral envelope vector is achieved specifically by dissolving the boron-containing compound in a solvent to form a solution and mixing this solution with a solution of the viral envelope vector. For incorporation of the boron-containing compound, a surfactant can also be preferably used. When the boron-containing compound is for example BSH, the boron-containing compound solution is mixed, in a ratio of from about 2 (HAU):1 (μg B) to 1 (HAU):2 (μg B) in terms of the amount of boron, with the viral envelope vector to which the various polymers were bound or not bound. Then, a surfactant-containing buffer is added to the mixture which is then centrifuged, followed by removing a supernatant, to give a BSH-enclosed viral envelope vector.

Such boron-containing compound preparation is used as it is or as a mixture with a pharmaceutically acceptable carrier, to serve as a drug that can be used particularly favorably in boron neutron capture therapy (BNCT).

The pharmaceutical preparation of the present invention can be used widely as it is or as a mixture with a pharmaceutically acceptable carrier, to work favorably in gene therapy, immunotherapy, chemotherapy, radiation therapy, and usual drug administration. The pharmaceutical preparation of the present invention is useful particularly in treatment of malignant pleural mesothelioma and osteosarcoma or in suppression of hepatic metastasis.

Therapy is conducted by administering the drug-enclosed preparation of the present invention via an any given suitable administration route in such a manner so as to allow the drug to be accumulated in a target site. The enclosed drug is preferably concentrated in a tumor. The drug-enclosed preparation can be administered all at once or in portions. Administration of the pharmaceutical preparation can be repeated as necessary. If desired, a tumor is excised surgically to the maximum degree and the remaining tumor is destroyed with the pharmaceutical preparation of the present invention.

Therapy with the boron-containing compound-enclosed preparation is conducted by administering the boron-containing compound-enclosed delivery vehicle via an any given suitable administration route in such a manner so as to allow the boron-containing compound to be accumulated in a target tumor, The compound is preferably concentrated in the tumor before irradiation, and the tumor:blood ratio before irradiation is advantageously about 2:1 or at least 1.5:1. The boron-containing compound-enclosed preparation can be administered all at once or in portions. After the compound is desirably accumulated in the tumor, an effective dose of low-energy neutron ray is applied to the site. The site can be irradiated through the skin, or the site can be completely or partially exposed before irradiation. Administration of the boron-containing compound and subsequent irradiation can be repeated as necessary. If desired, a tumor is excised surgically to the maximum degree and the remaining tumor is destroyed with the complex of the present invention. In another aspect, a suitable amount of the boron-containing compound is administered to a patient followed by irradiation of an effective dose of ²⁵²californium that is a naturally occurring neutron radioactive substance. Preferably, this is inserted into the tumor and removed at proper time.

For administration, the pharmaceutical preparation of the present invention can be mixed with a suitable excipient, an adjuvant and/or a pharmaceutically acceptable carrier and administered alone or in combination with another drug to a patient. A carrier that can be particularly preferably used is not limited and includes physiological saline, buffered physiological saline, dextrose, and water. In one embodiment of the present invention, the pharmaceutically acceptable carrier is pharmaceutically inert.

Administration of the drug of the present invention can be carried out orally or parenterally. In the case of parenteral administration, the drug can be administered arterially (for example, via a carotid artery), intramuscularly, subcutaneously, intramedullarly, intrathecally, intracerebroventricularly, intravenously, intraperitoneally or intranasally.

The pharmaceutical preparation can be in any forms such as powder, granules, micro granules, dry syrup, tablets, capsules, injection, and liquid medicine. The pharmaceutical preparation can be prepared by pharmaceutically known methods depending on the dosage form by suitably mixing with or diluting in/dissolving in pharmaceutical additives such as a suitable excipient; a disintegrating agent; a binder; a lubricant; a diluent; buffer agents such as phosphoric acid, citric acid, succinic acid, acetic acid, and other organic acids or salts thereof; a tonicity agent; a preservative; a wetting agent; an emulsifying agent; a dispersant; a stabilizer; a solubilizing agent; antioxidants such as ascorbic acid; a low-molecular (about less than 10 residues) polypeptide (for example, polyarginine or tripeptide); a protein (for example, serum albumin, gelatin, or immunoglobulin); a hydrophilic polymer (for example, polyvinyl pyrrolidone); an amino acid (for example, glycine, glutamic acid, aspartic acid, or arginine); monosaccharides, disaccharides and other carbohydrates (including cellulose or derivatives thereof, glucose, mannose or dextrin); a chelating agent (for example, EDTA); sugar alcohol (for example, mannitol or sorbitol); counter ions (for example, sodium); and/or nonionic surfactants (for example, polysorbate and poloxamer). Such substances enhancing isotonicity and chemical stability in the administration dose and concentration used are not toxic to the recipient.

Prescriptions and administration techniques are described for example in the latest edition and latest supplemental edition of Japanese Pharmacopoeia and in the final edition of Remington's Pharmaceutical Sciences, Maack Publishing Co., Easton, Pa.

The pharmaceutical preparation of the present invention is a drug contained in an amount effective for an intended drug to achieve an intended objective, and the term “therapeutically effective amount” or “pharmacologically effective amount” is sufficiently recognized by those skilled in the art and refers accurate dose is determined depending the severity of a disease in a patient to be treated (for example, the size and location of a tumor; the age, weight and sex of a patient; administration limited by diet time, administration frequency, drug combination, reaction susceptibility, and resistance/response to therapy).

Hereinabove, the present invention has been described by reference to preferable embodiments for facilitating understanding. Hereinafter, the present invention is described by reference to the Examples, but the above description and the following examples are provided for illustrative purposes only, and the scope of the present invention is limited neither to the embodiments nor to the Examples illustrated in this specification.

### Example 1

### (1) Proliferation of HVJ

A seed virus of HVJ was proliferated in a SPF (specific pathogen free) fertilized egg, separated and purified to give HVJ (Z seed) which was then pipetted into a tube for cell storage, supplemented with 10% DMSO, and stored in liquid nitrogen. Hen eggs just after fertilization were obtained, placed in an incubator (SHOWA-FURANKI P-03 type, which can accommodate about 300 hen eggs) and incubated at 36.5° C. under at least 40% humidity for 10 to 14 days. Survival of embryos, air spaces and chorioallantoic membranes were confirmed with an egg tester in to an amount of a drug effective in generating its pharmacological result, Determination of the therapeutically effective amount is sufficiently known to those skilled in the art.

The pharmaceutically effective amount refers to the amount of a drug that ameliorates a disease state by administration. Such therapeutic effect and toxicity of a compound can be determined by standard pharmacological procedures in cell culture or in experimental animals. The dose is preferably in the range of circulatory concentrations including ED50 accompanied by no or less toxicity. This dosage varies in this range depending on the administration form used, the susceptibility of a patient and the route of administration. By way of example, the amount of the complex administered can be selected suitably depending on the age and other conditions of a patient, the type of a disease, the type of the complex used, etc.

When the drug of the present invention is administered to a human, the drug corresponding to 400 to 400,000 HAD, preferably 1,200 to 120,000 HAD, more preferably 4,000 to 40,000 HAD, can be administered per subject.

“HAD” used herein refers to the activity of a virus that can agglutinate 0.5% of chicken erythrocytes. 1 HAU corresponds to nearly 24,000,000 viral particles (Okada, Y. et al., Biken Journal, 4, 209-213, 1961). The amount described above, for example, can be administered one to several times per day. The a dark room. The seed virus (recovered from liquid nitrogen) was diluted 500-fold with a polypeptone solution (a solution stored at 2 to 6° C. after preparation by mixing 1% polypeptone with 0.2% NaCl, adjusting its pH to 7.2 with 1 M NaOH, and sterilizing the solution in an autoclave) and then left to stand still at 2 to 6° C. Each egg was sterilized with Isodine and alcohol, and a small opening was formed in the egg with an eyeleteer Using a 1-ml syringe with a 26-gauge needle, 0.1 ml of the diluted seed virus was injected through the opening into the chorioallantoic cavity. Using a Pasteur pipette, melted paraffin (melting point 50 to 52° C.) was placed on the opening thereby clogging the opening. The egg was placed in an incubator and incubated at 34 to 36.5° C. under at least 40% humidity for 3 days. Then, the inoculated egg was left overnight at 2 to 6° C. The next day, the part of the air space in the egg was cut with tweezers, and a 10-ml syringe with an 18-gauge needle was inserted into the chorioallantoic membrane to suck up a chorioallantoic fluid which was then collected in a sterilized bottle and stored at 2 to 6° C.

### (2) Concentration of HVJ

About 100 ml of the HVJ-containing chorioallantoic fluid obtained in (1) above (HVJ-containing hen egg chorioallantoic fluid was collected and stored at 2 to 6° C.) was introduced via a wide-mouthed Komagome pipette into about 50-ml two centrifuge tubes and centrifuged at 3,000 rpm for 10 minutes at 2 to 6° C. in a low-speed centrifuge (with a brake off), to remove an egg tissue fragment. After centrifugation, the supernatant was pipetted into 35-ml four centrifuge tubes (for high-speed centrifugation) and centrifuged at 27,000 g for 30 minutes with an angle rotor. The supernatant was removed, and BSS (10 mM Tris-HCl (pH 7.5), 137 mM NaCl, 5.4 mM KCl; autoclaved and then stored at 2° C. to 6° C.) (PBS may be used in place of BSS) in an amount of about 5 ml per tube was added to the precipitate and left at 2′C to 6° C. overnight as it was. The precipitate was loosened gently by pipetting with a wide-mouthed Komagome pipette, collected in a 1 tube, and similarly centrifuged at 27,000 g for 30 minutes with an angle rotor. The supernatant was removed, and about 10 ml BSS was added to the precipitate and left similarly at 2° C. to 6° C. overnight. The precipitate was loosened gently by pipetting with a wide-mouthed Komagome pipette, and centrifuged at 3,000 rpm for 10 minutes at 2° C. to 6° C. in a low-speed centrifuge (with a brake off), thereby removing tissue fragments and viral aggregates that could have not been removed. The supernatant was introduced into a new sterilized tube and stored as an HVJ concentrate at 2° C. to 6° C. 0.9 ml of BSS was added to 0.1 ml of the HVJ concentrate and measured for its absorbance at 540 nm with a spectrophotometer, and the viral titer was converted into hemagglutination activity (HAU). An absorbance of 1 at 540 nm corresponded to nearly 15,000 HAU. HAU is considered almost proportional to fusion activity.

### (3) Preparation of HVJ Concentrate

Purification of HVJ with sucrose density gradient can also be conducted as necessary. Specifically, the HVJ suspension obtained in Example 1 was placed on layers of 60% and 30% sucrose solutions (sterilized) in a centrifuge tube and then subjected to density-gradient centrifugation at 62,800×g for 120 minutes. After centrifugation, a band seen on the 60% sucrose solution layer was recovered. The recovered HVJ suspension was dialyzed overnight against BSS or PBS to remove the sucrose. Unless used immediately, the HVJ suspension was supplemented with glycerol (autoclaved) and 0.5 M EDTA solution (autoclaved) at a final concentration of 10% and 2-10 mM respectively and frozen gently at −80° C. and stored finally in liquid nitrogen (in deep-freeze preservation, 10 mM DMSO may be used in place of glycerol and 0.5 M EDTA).

### Example 2

### Inactivation of HVJ by Irradiation with UV Ray

The purified and concentrated HVJ was irradiated with 99 mJ/cm² UV ray. The HVJ was dispensed into an Eppendorf tube (10,000 HAU/tube) and then centrifuged at 15,000 rpm for 15 minutes, and the precipitate was stored at −20° C.

Then, the inactivation of HVJ was evaluated. After inactivation treatment, the HVJ was used to infect simian renal cell strain LLC-MK2 cells at 37° C. for 1 hour, and 12 to 18 hours after infection with the HVJ, the cells were incubated at 37° C. for 18 to 24 hours in the presence of CO₂ gas and then fixed with acetone/methanol, and whether protein F of HVJ expressed in the HVJ-infected cells occurred or not was examined by immunostaining with an antibody to protein F. That is, HVJ was solubilized with a surfactant NP-40 (nonylphenoxypolyethoxyethanol) and centrifuged to separate a membrane component, and the resulting membrane component was subjected to ion-exchange chromatography to give protein F (according to Yoshima, H., et al., J. Biol. Chem. 1981, and Suzuki, K., at al., Gene Therapy and Regulation, 2000). Then, this protein F, together with Freund's adjuvant, is used to immunize a rabbit four times to give an antiserum to protein F (rabbit protein F polyclonal antibody: primary antibody). The fixed cells were treated with this primary antibody for 1 hour and then treated with a FITC-labeled porcine anti-rabbit IgG polyclonal antibody (secondary antibody) for 1 hour. After this treatment with the secondary antibody, the cells can be observed under a fluorescence microscope to evaluate the inactivation of HVJ. The influence of the inactivation treatment on the membrane function of the virus (for example HVJ) envelope can be examined by measuring, as an indicator, the HA activity of the inactivated virus (for example HVJ) envelope. The HA activity can be measured by a usual method. A suspension of the inactivated HVJ envelope was added in amounts of 50, 40 and 30 μl respectively to 3 wells of a 96-well plate (round bottom) and then serially diluted twofold with PBS (−) (Mg ion- and Ca ion-free Dulbecco's phosphate buffered saline) to prepare serially diluted samples. PBS (−) containing 0.5% hen erythrocytes was added thereto and incubated at 2° C. to 6° C. for 2 hours, and whether the agglutination reaction occurred or not was examined. The HA activity was determined from the amount of the serially diluted sample in which the agglutination reaction was lost and a reciprocal of the degree of dilution in the corresponding well.

### Example 3

### Purification of Inactivated HVJ by Column Chromatography and Ultrafiltration

### (1) Purification by Column Chromatography

The inactivated HVJ solution obtained in Example 2 was fed at a flow rate of 50 mL/min. to a Q-Sepharose FF column (diameter 20 cm, bed height 15 cm, bed volume 4710 ml) previously equilibrated with 15-L buffer 1 (20 mM Tris-HCl (pH 7.5), 150 mM NaCl). Then, 10-L buffer 1 (20 mM Tris-HCl (pH 7.5), 150 mM NaCl) and 25-L buffer 2 (20 mM Tris-HCl (pH 7.5), 350 mM NaCl) were passed in this order through the column. When the concentrate was fed, the inactivated HVJ was adsorbed on the column resin, while a majority of impurities in the inactivated HVJ concentrate were washed away from the resin with the buffers 1 and 2. When 25-L buffer 3 (20 mM Tris-HCl (pH 7.5), 650 mM NaCl) was passed, HVJ was eluted at almost the same time from the resin, so collection of column fractions was initiated. A peak of inactivated HVJ appeared on a UV absorption chart (λ=280 nm), and while the peak returned to the baseline, a 7829-mL fraction was obtained. An antibiotic was added to this fraction. After fractionation, passage of the buffer was continued, and finally 20-L buffer 4 (20 mM Tris-HCl (pH 7.5), 1 M NaCl) was passed through the column.

### (2) Purification by Ultrafiltration

The column fraction obtained in step (1) in Example 3 was introduced into a 10-L bottle which was then tightened with a cap to which a liquid-feeding tube and a circulatory tube had been attached. The liquid-feeding tube was connected via a peristaltic pump to an inlet of UFP-500-E-5A ultrafiltration module manufactured by A/G Technology Corporation, and the circulatory tube was connected via a circulating volume-regulating valve to an outlet of the module. The pump was operated, and the sample was concentrated by narrowing down the circulating volume-regulating valve while the pressure at the outlet side of the module was kept at 40 to 80 kPa, to drain fluid at a rate of 60 to 70 mL/min. After the circulating volume reached about 600 mL, the bottle was exchanged with a 500-mL bottle, and the module was exchanged with UFP-500-E-4A manufactured by A/G Technology Corporation, to continue concentration. Fluid was drained at a rate of about 10 mL/min. in the same manner as above, and after the circulating volume reached about 60 mL, 60 mL buffer 5 (20 mM Tris-HCl (pH 7.5) 50 mM NaCl, 1 mM MgCl₂, 2% mannitol) was added, and concentration was further continued so that the circulating volume reached about 60 mL (buffer was exchanged). After buffer exchange was carried out further twice, the circulating volume was 79 mL. The circulating fluid was taken into a 5-mL disposable syringe, and a disk filter (Sterile Syringe φ=26 mm, 0.45 μm, manufactured by CORNING) was attached to the top of the syringe, thorough which sterilization filtration was manually conducted.

### Example 4

### Production of Cationized Gelatin

Low-molecular-weight gelatin with a molecular weight of 5,000 was mixed with ethylene diamine in an amount of 50-mole equivalent of carboxyl groups in the gelatin, and the mixture was added to 0.1 M phosphate buffer, pH 5, and reacted overnight at 37° C. in the presence of EDC. After dialysis, the sample was dried to give cationized gelatin.

### Example 5

### Production of Hyaluronic Acid-Bound (or -Introduced) Cationized Gelatin

The cationized gelatin prepared in Example 4 and hyaluronic acids having various molecular weights were added to 0.2 M carbonate buffer, pH 9.7, and stored at 37° C. for 3 days in the presence of NaCNBH₃, thereby reacting the sugar reducing terminal with an amino group of the cationized gelatin. After dialysis, the sample was dried to give hyaluronic acid-bound (or -introduced) cationized gelatin. The prepared cationized gelatin, and hyaluronic acids having various molecular weights, were added to 0.2 M carbonate buffer, pH 9.7, and stored at 37° C. for 3 days in the presence of NaCNBH₃, thereby reacting the sugar reducing terminal with an amino group of the cationized gelatin. After dialysis, the sample was dried to give hyaluronic acid-bound (or -introduced) cationized gelatin. The hyaluronic acids having various molecular weights had been obtained by thermally decomposing hyaluronic acid having a molecular weight of about 1,800,000 in an autoclave, then dialyzing and purifying the product. In the above procedure, hyaluronic acids having a molecular weight of 5,000, and cationized gelatin having a molecular weight of 3,100, were mixed in a ratio of 1:1.

### Example 6

### Method of Preparing Polyethylene Glycol-Bound (or -Introduced) Cationized Gelatin

The cationized gelatin prepared in Example 4 and polyethylene glycols having various molecular weights were added to 0.2 M carbonate buffer, pH 9.7, and stored at 37° C. for 1 hour in the presence of NaCNBH₃, thereby reacting an aldehyde group at the terminal of the polyethylene glycol with an amino group of the cationized gelatin. After dialysis, the sample was dried to give polyethylene glycol-bound (or -introduced) cationized gelatin. The molecular weights of the respective polyethylene glycols range from about 10 kDa (kilodalton) to 100 kDa. Using the molecular weight and the degree (1 to 20%) of inclusion of the polyethylene glycols as parameters, polyethylene glycols having the respective molecular weights were separated before use. In initial setting, PEG having a molecular weight of 5,000 and cationized gelatin having a molecular weight of 3,100 were mixed in ratios of 1:1, 5:1, and 10:1.

### Example 7

### Preparation of Polymer-Bound HVJ-E

The polymer-bound HVJ-E was prepared as follows by compounding the various polymers obtained in Examples 4, 5 and 6 with the HVJ-E obtained in Example 1 basically in a ratio of 250 μg to 500 HAU, 50 μL of the various polymer solutions at 20 mg/ml (in PBS as the solvent) were prepared, and 40 μL of 4 samples (HVJ-E stock, 500 HAU/10 μL) were added to the polymers respectively and pipetted (total volume of 90 μL). Then, 110 μl PBS was added thereto (total volume of 200 μL) and left to stand still for 30 minutes on ice, thereby HVJ-E to which the various polymers had been added were prepared.

### Example 8

### Evaluation of Mouse Toxicity

The HVJ-E obtained in Example 1 and the polymer-bound HVJ-E obtained in Example 7 were used in a mouse toxicity test. HVJ-E, and the various polymers-bound HVJ-E preparations (GC 1,000 mg: 2,000 HAU) were dissolved in PBS to a final volume of 200 mL, and then administered to each of the heart chambers of normal C57/BL6 mice, and from the number of mice that have survived for 1 week or more after administration, the survival rate was calculated. The results are shown in Table 1.

TABLE 1

| Amount | | | | |
|---|---|---|---|---|
| of HVJ-E | HVJ-E | CG-HVJ- | CG-PEG- | CG-HA- |
| (HAU) | alone | E | HVJ-E | HVJ-E |
| 1,000 | 100% (3/3) | | | |
| 1,500 | 100% (3/3) | | | |
| 2,000 | 40% (2/5) | 100% (3/3) | 100% (3/3) | 100% (1/1) |
| 2,500 | | 67% (2/3) | 100% (2/2) | 100% (2/2) |
| 3,000 | 33% (1/3) | 0% (0/1) | 0% (0/3) | 50% (1/2) |
| 4,000 | 0% (0/3) | | 0% (0/2) | |
| 5,000 | 0% (0/3) | | | |
| 6,000 | | | 0% (0/1) | |

In Table 1, the maximal permissible doses of the various polymers-HVJ-M at which all the mice could survive were 1,500 HAU for HVJ-E, 2,000 HAU for CG-HVJ-E, 2,500 HAU for CG-PEG-HVJ-E and 2,5000 HAU for CG-HA-HVJ-E, and it was recognized that the toxicity was lower in the polymer-bound HVJ-E, that is, the complexes conjugated with PEG and hyaluronic acid obtained respectively in Examples 5 and 6, than in the HVJ-E obtained in Example 1 and CG-HVJ-E obtained in Example 4.

### Example 9

### Evaluation of Inhibitory Effect on Blood Agglutination

The HVJ-E obtained in Example 1, and the polymer-bound HVJ-E suspension (CG 250 mg: 500 HAU) obtained in Example 7, were serially diluted twofold to prepare serially diluted samples respectively, and 150 mL was added to a well plate, and whether the agglutination reaction occurred or not was examined. 20 μl of the sample was added to each well of a 96-well microtiter plate, and a test solution in which 1 ml blood obtained by collection of blood from a human had been suspended in 49 ml of physiological saline was added in an amount of 90 μl/well to the plate and left to stand still at room temperature for 2 hours, and the minimum concentration at which erythrocyte agglutination activity was observed with the naked eye was judged. The HA activity was determined from the amount of the serially diluted sample in which the agglutination reaction was lost and a reciprocal of the degree of dilution in the corresponding well. As a result, it was recognized that the polymer-bound HVJ-E obtained in Example 7 exhibited a twofold blood agglutination inhibitory action in vitro as compared with the HVJ-E obtained in Example 1.

### Example 10

### Inclusion of a Drug

A solution of BSH at 17,240 μgB/ml, that is 7,240 μg (in terms of boron atom B) per ml of PBS as the solvent, was prepared. This solution was used to prepare a pharmaceutical preparation wherein the mixing ratio of the various polymers-bound HVJ-E obtained in Example 7 to BSH was 1,500 HAU 1,000 μg B in BSH. 6666.7 μg B (386.7 μl) in BSH was added to 10,000 HAU polymer-bound HVJ-E per tube, followed by sufficient pipetting. Then, 40 μl of 3% Triton X-100/TE buffer solution was added to each tube, then vortexed, left to stand still for 5 minutes on ice, and centrifuged at 15000 rpm/4° C./5 min. 1.0 ml of BSS solution was added to each tube, vortexed and centrifuged at 15000 rpm/4° C./5 min., to remove a supernatant, whereby the sample was purified.

### Example 11

### Evaluation of the Efficiency of Introduction of Luciferase

CD44-expressing LM8G5 (mouse osteosarcoma cell strain: hepatic metastasis high expression strain established by in vivo selection from LM8G5 purchased from RIKEN CELL BANK) was suspended in 10% fetal bovine serum-containing RPMI 1640 culture medium at a density of 1×10⁴ cells/0.2 mL/well (24-well plastic plate) and cultured at 37° C. in a 5% CO₂ gas incubator. After culture for 20 to 24 hours, the cells were subjected to measurement of introduction of a gene with HVJ-E. Similarly, CT26 (human colon cancer cell strain purchased from ATCC) was suspended in 10% fetal bovine serum-containing RPMI 1640 culture medium at a density of 1×10⁴ cells/0.2 mL/well (24-well plastic plate) and cultured at 37° C. in a 5% CO₂ gas incubator. After culture for 20 to 24 hours, the cells were subjected to measurement of introduction of a gene with HVJ-E.

5 μL of 2 mg/mL protamine sulfate solution (in PBS) was added to, and mixed with, the HVJ-E obtained in Example 1 or each of the various polymers-bound HVJ-E suspensions (in PBS as the solvent) obtained in Example 7, and the mixture was left to stand still on ice for 5 minutes. Subsequently, 5 μL (10 μg) of a solution of a plasmid DNA (pGL3) harboring a luciferase gene was added to, and mixed with, the above solution, and 3 μL of 2% Triton X-100 (PBS (−)) was further added thereto, and the mixture was centrifuged at 15000 rpm (19500×G) at 2° C. to 6° C. for 10 minutes to 15 minutes. After the supernatant was removed, the precipitate was suspended with 30 μl PBS (−). 5 μL of 1 mg/mL protamine sulfate solution (in PBS) was added to, and mixed with, the suspension. This mixture was added in an amount of 8 μL (per well) to previously prepared (cultured) LM8G5 cells or CT26 cells.

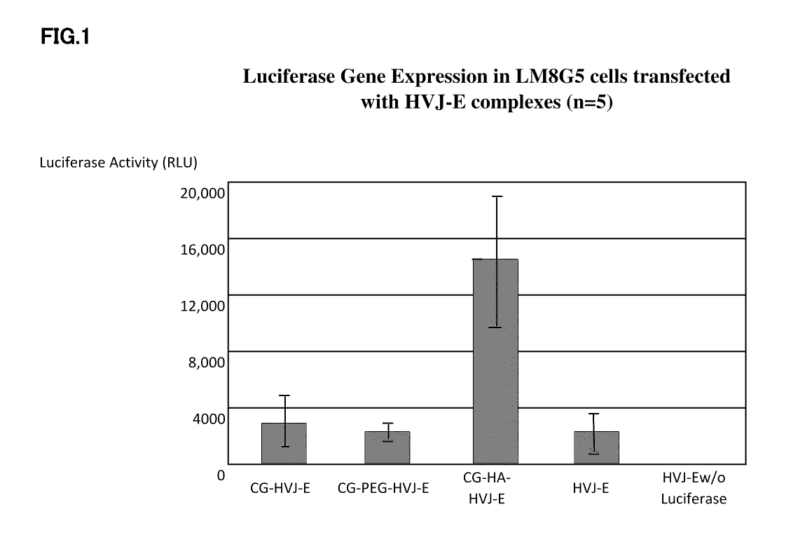

At 20 to 24 hours after addition, the expression level of luciferase was measured with a luciferase measurement kit (LucLite, No. 6016911, manufactured by Packard). The emission was measured with a luminometer (TD-20e LUMINOMETER manufactured by Turner). The results are shown in FIG. 1. As is evident from FIG. 1, the HVJ envelope was used to demonstrate that particularly a biological polymer such as a gene can be introduced into LM8G5. This tendency was significant when the hyaluronic acid-introduced cationized HVJ-E was used.

### Example 12

### Evaluation of Affinity of HVJ-E for Tumor Cells

By the same method as in Example 11, LM8G5 cells were maintained. Separately, a fluorescent dye Qdot 655 (Qd) was enclosed in the HVJ-E obtained in Example 1 and in the various polymers-bound HVJ-E obtained in Example 7. This enclosure was carried out in the same manner as in Example 10. LM8G5 cells were contacted with each of the viral envelope vectors for 1 hour at normal temperature, then washed and cultured for 24 hours, and the binding of each viral envelope vector to the cells was observed under a fluorescence microscope to examine the affinity of the respective viral envelope vectors for the tumor cells. As a result, particularly strong affinity for the tumor cells was recognized in the hyaluronic acid-introduced cationized HVJ-E.

### Example 13

### BNCT Irradiation Experiment In Vitro with BSH-Enclosed HVJ-E Vector Preparation

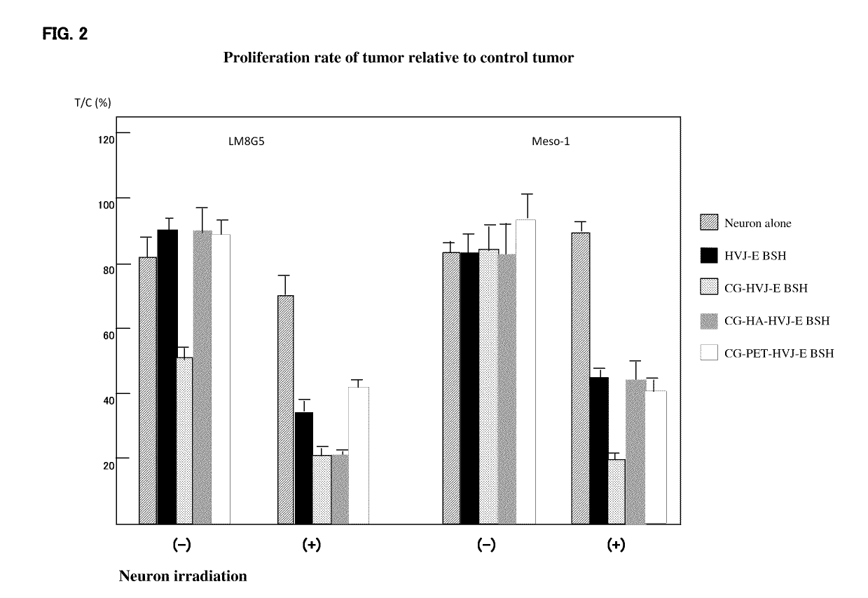

Each of the various BSH-enclosed HVJ-E vectors obtained in Example 10 was added to a mouse osteosarcoma cell strain LM8G5 culture and a human malignant pleural mesothelioma cell strain MESO-1 culture, respectively, and left for 10 minutes and then irradiated directly with neutrons for 1 hour, and the cells were cultured for 1 week, and whether there was the cell growth inhibitory effect was examined. As a result, the cell growth inhibitory effect was confirmed in any cells depending on the concentration of boron by neutron irradiation as shown in FIG. 2.

### Example 14

### BNCT Irradiation Experiment In Vivo with BSH-Enclosed HVJ-E Vector Preparation

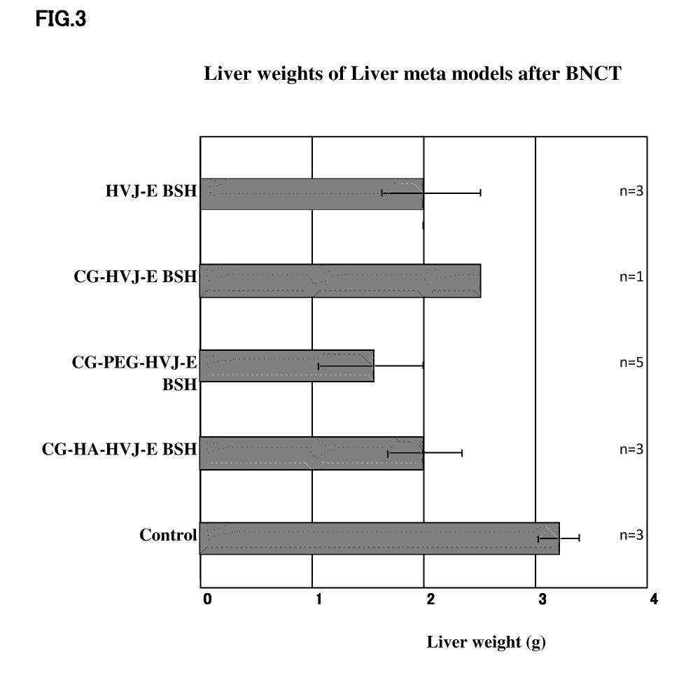

The antitumor effects of the various BSH-enclosed HVJ-E vectors obtained in Example 10 were examined by using a mouse osteosarcoma cell strain hepatic metastasis model. First, each of C3H/HeN mouse was inoculated via a superior mesenteric vein with 1×10⁶ LM8G5 cells on Day 0. On Day 8, each of the various BSH-enclosed HVJ-E vectors, or BSH (dissolved in PBS) only as the control, was introduced into the heart chamber of each mouse. The amount of the introduced BBB in each sample, in terms of boron ¹⁰B, was 1,000. The mice were kept for 1 day and then treated by irradiation with neutrons. Irradiation was carried out for 60 minutes per day. On Day 11, the mice were sacrificed, and after treatment, the liver was removed from each mouse, and the weight of the liver was measured thereby examining the therapeutic effect of the BSH-enclosed HVJ-E vectors of the present invention. As a result, it was found as shown in FIG. 3 that any BSH-enclosed HVJ-E vectors had an antitumor effect by neutron irradiation, and particularly a strong antitumor cell effect was recognized in PEG-introduced cationized HVJ-E.

Then, the antitumor effects of the various BSH-enclosed HVJ-E vectors obtained in Example 10 were examined by using a mouse pleuritic model. First, 5×10⁶ MESO-1 cells were injected into the right thoracic cavity of each of C3H/HeN mice on Day 0. On Day 7 to Day 14, each of the various BSH-enclosed HVJ-E vectors, or BSH only as the control, was introduced into the thoracic cavity. The amount of the introduced BSH in each sample, in terms of boron ¹⁰B, was 1,000. The mice were kept for 1 day and then treated by irradiation with neutrons. Irradiation was carried out for 60 minutes per day. On Day 8 to Day 15, the mice were sacrificed, and after treatment, the thorax of each mouse was opened, and the state of each of pleura was examined thereby examining the therapeutic effect of each of the BSH-enclosed HVJ-E vectors of the present invention, As a result, it was found that any BSH-enclosed HVJ-E vectors had an antitumor effect by neutron irradiation.

1-13. (canceled)

14. A method for treating cancer with boron neutron capture therapy (BNCT) comprising:
(i) administering to a patient of cancer,
a drug delivery vehicle comprising
a viral envelope vector, which is cationized by hyaluronic acid-bound cationized gelatin and/or polyethylene glycol-bound cationized gelatin, and
a boron-containing compound enclosed in the vehicle; and
(ii) providing neutron irradiation to the patient.

15. The method of claim 1, wherein the cancer is malignant mesothelioma of pleura or osteogenic sarcoma.

16. The method of claim 1, wherein the boron-containing compound is mercaptoundecahydrododecaborate (BSH) or p-boronophenylalanine (BPA).

17. The method of claim 1, wherein the viral envelope vector is HVJ-E derived from a Sendai virus.
